(19) United States
(12) Patent Application Publication
(10) Pub. No.: US 2026/0229314 A1
(43) Pub. Date: Aug. 6, 2026

(54) SYSTEM FOR INSTRUMENT OPTIMIZATION USING ANALYTE BASED MASS SPECTROMETER AND ALGORITHM PARAMETERS

(71) Applicants: Thermo Finnigan LLC, San Jose, CA (US); FEI Company, Hillsboro, OR (US)

(72) Inventors: Scott Ryan Kronewitter, Waltham, MA (US); Sylvester Greer, Round Rock, TX (US); Paul R Gazis, Sunnyvale, CA (US); Weijing Liu, San Jose, CA (US); Albert Konijnenberg, Zaltbommel (NL); Roza I Viner, Fremont, CA (US); Ping F Yip, Salem, MA (US)

(21) Appl. No.: 19/147,813

(22) PCT Filed: Jan. 15, 2024
(86) PCT No.: PCT/US2024/011557

§ 371 Date: Jul. 14, 2025

Related U.S. Application Data
(60) Provisional application No. 63/480,081, filed on Jan. 16, 2023.

(51) Int. Cl. G16B 40/10

(52) U.S. Cl. CPC G16B 40/10

(57) ABSTRACT

Disclosed herein are scientific instrument support systems, as well as related methods, computing devices, and computer-readable media. For example, in some embodiments, an apparatus, includes a sample introduction device and mass spectrometer. The mass spectrometer includes first logic to receive a sample from a queue, the sample comprising an analyte; second logic to determine instrument methods and data analysis parameters based on a nature of the analyte; and third logic to process the sample by applying the instrument methods and data analysis parameters. WO

### CROSS-REFERENCE TO RELATED APPLICATION

This application claims priority to U.S. Provisional No. 63/480,081, filed on Jan. 16, 2023, titled “SYSTEM FOR INSTRUMENT OPTIMIZATION USING ANALYTE BASED MASS SPECTROMETER AND ALGORITHM PARAMETERS,” the entire content of which is incorporated herein by reference.

### BACKGROUND

Chromatography is a technique for the separation of constituents of a sample mixture that utilizes the differing properties of the constituents as they interact with other materials. Mass spectrometry is a technique for detecting, identifying, and quantifying molecules within samples based on their molecular mass-to-charge ratio after ionization.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, not by way of limitation, in the figures of the accompanying drawings.

FIG. 1 is a block diagram of an example mass spectrometry instrument support module, in accordance with various embodiments.

FIG. 2 is a flow diagram of an example method to perform support operations, in accordance with various embodiments.

FIG. 3 is an example of a graphical user interface that may be used in the performance of some or all of the support methods disclosed herein, in accordance with various embodiments.

FIG. 4 is a block diagram of an example computing device that may perform some or all of the mass spectrometry instrument support methods disclosed herein, in accordance with various embodiments.

FIG. 5 is a block diagram of an example mass spectrometry instrument support system in which some or all of the mass spectrometry instrument support methods disclosed herein may be performed, in accordance with various embodiments.

FIG. 6 is an example of a visual indicator marking sample quality determinations generated using the instrument control and data analysis techniques disclosed herein for an array of samples disposed in wells of a tray, in accordance with various embodiments.

### DETAILED DESCRIPTION

Disclosed herein are scientific instrument support systems, as well as related methods, computing devices, and computer-readable media. For example, in some embodiments, a mass spectrometry instrument support apparatus may include: first logic to receive a sample from a queue, the sample comprising an analyte; second logic to determine instrument methods and data analysis parameters based on a nature of the analyte; and third logic to process the sample by applying the instrument methods and data analysis parameters for an elution time where the mass is predicted as a function of elution time.

Proteins and protein complexes have been studied with mass spectrometry for decades. Accordingly, there is a need for democratizing mass spectrometry so that they are easier to use, and the complexities are calibrated automatically, without user intervention. Moreover, applying the correct parameters for a given mass range dramatically improves the sensitivity and specificity of the analysis for protein complexes and other large masses (e.g., 50 Kilodalton (KDa)-1000 KDa and greater).

As discussed in further detail below, size exclusion chromatography or lower resolution online buffer exchange separate molecules based on size where larger molecules pass through first and smaller molecules, which are slowed down by pore interactions, pass through later. As such, in some embodiments, the described system employs this separation characteristic to determine when different sized molecules will elute from the columns and set methods and parameters accordingly.

Generally, mass spectrometers have a variety of settings that allow for optimizing the instrument to improve analysis of, for example, a certain mass range. These settings include, for example, hardware settings (e.g., temperatures, pressures), ion optics (e.g., voltages, trapping times), and detector parameters (e.g., transient lengths, mass calibration). In some embodiments, these settings can be pre-optimized for a mass range and applied as an applied in-sync with the different masses coming off the column.

Additionally, data analysis algorithms generally do not apply well uniformly across all masses because other factors besides varying parameters are frequently desired. For example, often different algorithms are needed with regards to noise filtering, feature detection, mass deconvolution, mass clustering, and the like. In such examples, parameters assigned as a function of mass and the algorithms are also selected.

Accordingly, the mass spectrometry instrument support embodiments disclosed herein may include applying pre-determined, optimal parameters for when a known mass is received. In some embodiments, these parameters are applied for an elution time where the mass is predicted as a function of elution time (e.g., retention time). Accordingly, the scientific instrument embodiments disclosed herein may achieve improved performance relative to conventional approaches. Specifically, embodiments may be employed for screening samples for cryogenic electron microscopy (cryo-EM) improving the yield of good structures per sample. For example, bad samples can be screened or triaged to send only the highest quality samples to the vitrification and cryo-EM.

In some embodiments, the described system is employed to analyze a wide range of compounds by coupling size-based separations (including on-line desalting), mass spectrometry, and data analysis. In some embodiments, the system leverages a separation mode of the column chemistry to automatically select optimized instrument methods and data analysis parameters based on the character of the analyte. For example, the instrument methods and data analysis parameters may be selected to match the analyte's size to improve fidelity analysis of the samples at the system level. Thus, the described system avoids the typical pitfalls of manual selection and therefore collecting and analyzing data under sub-optimal conditions. In some embodiments, the system is deployed onto the mass spectrometer to collect samples from a queue, which are then analyzed without user intervention.

The automated software system described here stores all the settings (e.g., instruments, methods, parameters, algorithms) into a collection of “automatable units” that are cached until needed. As the samples are acquired by the instrument, the automatable units are triggered automatically upon completion of the run. In some embodiments, the parameters are applied, and the data analysis performed to calculate sample quality and sample purity. In some embodiments, a stop light color system (e.g., red, yellow, green) is employed, in addition to numerical quality metrics, to mark sample quality in an easy to consume visual. In some embodiments, the color system and metrics can be used for quality control samples to indicate the performance of the instrument on known standard samples. The system allows for large scale, automated, applications for screening and quality control by, for example, automatically analyzing data and displaying sample quality.

As further discussed herein, some embodiments of the systems and methods disclosed herein may generate instrument control and data analysis parameters based on the mass of the analyte of interest. Using mass as the value from which to set parameters may be particularly advantageous when mass spectrometry is combined with size exclusion chromatography or online buffer exchange, as these methods separate constituents according to their mass, and thus provide well-characterized results for input to a mass-based mass spectrometry parameter determination system.

In the following detailed description, reference is made to the accompanying drawings that form a part hereof wherein like numerals designate like parts throughout, and in which is shown, by way of illustration, embodiments that may be practiced. It is to be understood that other embodiments may be utilized, and structural or logical changes may be made, without departing from the scope of the present disclosure. Therefore, the following detailed description is not to be taken in a limiting sense.

Various operations may be described as multiple discrete actions or operations in turn, in a manner that is most helpful in understanding the subject matter disclosed herein. However, the order of description should not be construed as to imply that these operations are necessarily order dependent. In particular, these operations may not be performed in the order of presentation. Operations described may be performed in a different order from the described embodiment. Various additional operations may be performed, and/or described operations may be omitted in additional embodiments.

For the purposes of the present disclosure, the phrases “A and/or B” and “A or B” mean (A), (B), or (A and B). For the purposes of the present disclosure, the phrases “A, B, and/or C” and “A, B, or C” mean (A), (B), (C), (A and B), (A and C), (B and C), or (A, B, and C). Although some elements may be referred to in the singular (e.g., “a processing device”), any appropriate elements may be represented by multiple instances of that element, and vice versa. For example, a set of operations described as performed by a processing device may be implemented with different ones of the operations performed by different processing devices.

The description uses the phrases “an embodiment,” “various embodiments,” and “some embodiments,” each of which may refer to one or more of the same or different embodiments. Furthermore, the terms “comprising,” “including,” “having,” and the like, as used with respect to embodiments of the present disclosure, are synonymous. When used to describe a range of dimensions, the phrase “between X and Y” represents a range that includes X and Y. As used herein, an “apparatus” may refer to any individual device, collection of devices, part of a device, or collections of parts of devices. The drawings are not necessarily to scale.

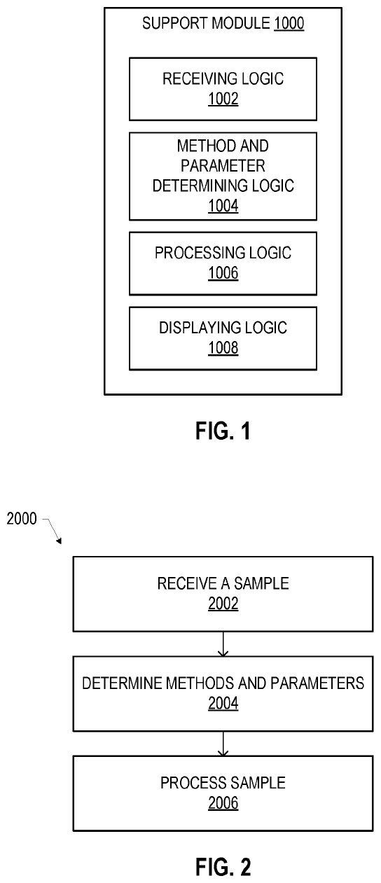

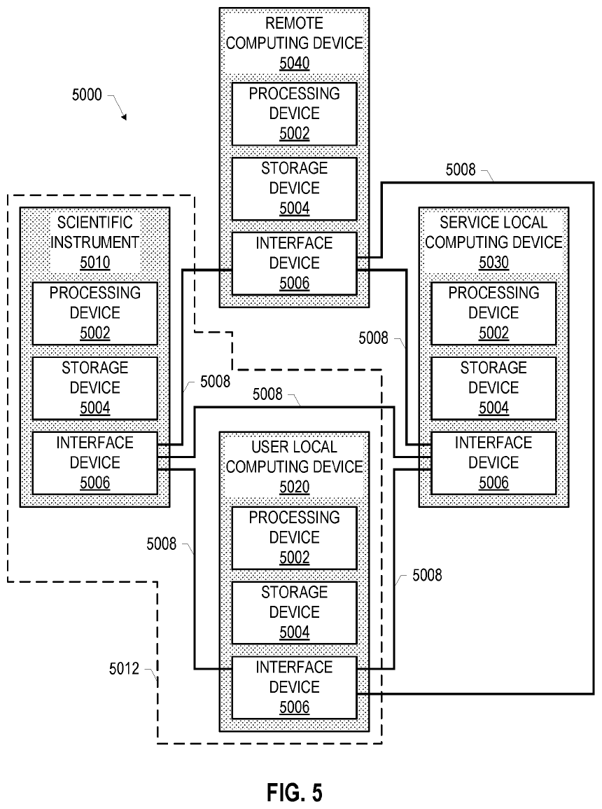

FIG. 1 is a block diagram of a mass spectrometry instrument support module 1000 to determine when different sized molecules will elute from the columns and set methods and parameters accordingly. The mass spectrometry instrument support module 1000 may be implemented by circuitry (e.g., including electrical and/or optical components), such as a programmed computing device (e.g., a device for measuring the mass). The logic of the mass spectrometry instrument support module 1000 may be included in a single computing device or may be distributed across multiple computing devices that are in communication with each other as appropriate. Examples of computing devices that may, singly or in combination, implement the mass spectrometry instrument support module 1000 are discussed herein with reference to the computing device 4000 of FIG. 4, and examples of systems of interconnected computing devices, in which the mass spectrometry instrument support module 1000 may be implemented across one or more of the computing devices, is discussed herein with reference to the mass spectrometry instrument support system 5000 of FIG. 5. The mass spectrometry instrument support module 1000 may include sample receiving logic 1002, method and parameter determining logic 1004, processing logic 1006, and displaying logic 1008.

As used herein, the term “logic” may include an apparatus that is to perform a set of operations associated with the logic. For example, any of the logic elements included in the support module 1000 may be implemented by one or more computing devices programmed with instructions to cause one or more processing devices of the computing devices to perform the associated set of operations. In a particular embodiment, a logic element may include one or more non-transitory computer-readable media having instructions thereon that, when executed by one or more processing devices of one or more computing devices, cause the one or more computing devices to perform the associated set of operations. As used herein, the term “module” may refer to a collection of one or more logic elements that, together, perform one or more functions associated with the module. Different ones of the logic elements in a module may take the same form or may take different forms. For example, some logic in a module may be implemented by a programmed general-purpose processing device, while other logic in a module may be implemented by an application-specific integrated circuit (ASIC). In another example, different ones of the logic elements in a module may be associated with different sets of instructions executed by one or more processing devices. A module may not include all of the logic elements depicted in the associated drawing; for example, a module may include a subset of the logic elements depicted in the associated drawing when that module is to perform a subset of the operations discussed herein with reference to that module.

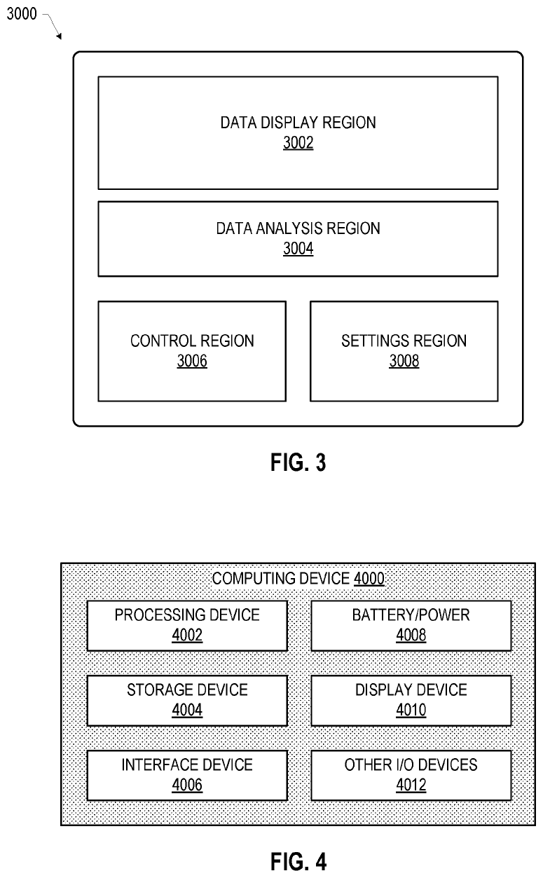

The sample receiving logic 1002 may be configured to receive a sample from a queue. In some examples, sample includes an analyte. The sample receiving logic 1002 may receive samples on a regular chronological schedule (e.g., a set number of seconds or minutes), after a certain number of samples accumulate in the queue (e.g., 20), in accordance with any other suitable schedule, or at the command of a user (e.g., received via a GUI, such as the GUI 3000 of FIG. 3).

The method and parameter determining logic 1004 may be configured to determine instrument methods and data analysis parameters based on a nature of the analyte. In some examples, the nature of the analyte includes size, mass, shape, structure, or chemical composition. In particular, in certain embodiments, a user may specify a mass (i.e., a molecular weight) of an analyte of interest, or may specify a chemical structure of the analyte of interest (e.g., in the form of a FASTA file or other format for describing nucleotide or protein sequences). When a user specifies a chemical structure of an analyte of interest(e.g., by selecting or otherwise pointing to a FASTA or other appropriate file), the logic 1004 may calculate the mass of the analyte of interest based on the chemical structure using known techniques, and then may determine instrument methods and data analysis parameter based on the calculated mass.

As noted above, the logic 1004 may determine instrument methods (e.g., instrument parameters) based on the nature of the analyte (e.g., the mass of the analyte, as specified by a user). In some examples, the parameters include hardware settings, ion optics, or detector parameters. In some particular embodiments, the instrument parameters may include scan range (specified as an m/z range), a desolvation voltage, a trapping gas setting, or a resolution. In one example of such an embodiment, the logic 1004 may determine that: for an analyte whose molecular weight is between 0 and 50 kDa, the scan range will be 1500-6000 m/z, the desolvation voltage may be 50 V, the trapping gas setting may be 3, and the resolution may be 100000; for an analyte whose molecular weight is between 50 and 300 kDa, the scan range will be 2500-10000 m/z, the desolvation voltage may be 100 V, the trapping gas setting may be 5, and the resolution may be 12500; for an analyte whose molecular weight is between 300 and 700 kDa, the scan range will be 5000-20000 m/z, the desolvation voltage may be 100 V, the trapping gas setting may be 6, and the resolution may be 6250; and for an analyte whose molecular weight is between 700 and 1000 kDa, the scan range will be 6000-24000 m/z, the desolvation voltage may be 100 V, the trapping gas setting may be 7, and the resolution may be 3125.

As noted above, the logic 1004 may determine data analysis parameters (e.g., which algorithms to perform to analyze data from the instrument, and/or which parameters to use with the selected algorithm) based on the nature of the analyte (e.g., the mass of the analyte, as specified by a user). For example, in some embodiments, the logic 1004 may determine which of different algorithm options to choose for different data analysis processes, such as noise filtering, feature detection, mass deconvolution, and mass clustering, among others. The logic 1004 may select from any suitable known algorithms for different ones of these processes.

In some embodiments, the logic 1004 may utilize mass information to determine which algorithms to select. For example, the logic 1004 may select from different available mass deconvolution algorithms (e.g., select from the “Zscape” algorithms described in U.S. Pat. No. 10,217,619, “Methods for data-dependent mass spectrometry of mixed intact protein analytes” or the “BCDecon” algorithms described in U.S. patent application Ser. No. 18/337, 183, “Bayesian decremental scheme for charge state deconvolution”). In a particular example, for an analyte with a molecular weight between 0 and 50 kDa (with “high-resolution” data in which the mass spectra contain features where the compound isotopomers are fully or partially resolved), the logic 1004 may select certain algorithms (e.g., a Zscape algorithm for mass deconvolution), and for an analyte with a molecular weight between 50 and 1000 kDa (with “low-resolution data” in which where the features contain compound isotopomers that are not resolved, yet the charge states are still resolvable), the logic 1004 may select other algorithms (e.g., a BCDecon algorithm for mass deconvolution).

In some embodiments, the logic 1004 may utilize mass information to set the parameters for one or more selected algorithms. The association between the mass of an analyte of interest and the appropriate parameters may be stored in a memory available to the logic 1004 so that the logic 1004 may generate or identify the appropriate parameters in response to a specified mass. The values of the appropriate parameters may be generated by routine experimentation for different types of analytes and sets of conditions, and stored for access by the logic 1004. For example, for mass deconvolution, examples of parameters whose values may be selected based on the mass of an analyte of interest may include the parameters of the Zscape algorithms, the parameters of the BCDecon algorithms, or the parameters of any other suitable mass deconvolution algorithm.

The processing logic 1006 may be configured to process the sample by applying the instrument methods and data analysis parameters for an elution time where the mass is predicted as a function of elution time. In some cases, the sample is processed by coupling size-based separations. In some examples, the parameters are pre-optimized for a mass range and applied as an applied in-sync with the different masses coming off a column.

The displaying logic 1008 may provide to a user, through a GUI (such as the GUI 3000 of FIG. 3), an option to view the results of the processing logic 1006 or select a sample from the queue for the sample receiving logic 1002.

FIG. 2 is a flow diagram of a method 2000 of performing support operations, in accordance with various embodiments. Although the operations of the method 2000 may be illustrated with reference to particular embodiments disclosed herein (e.g., the scientific instrument support modules 1000 discussed herein with reference to FIG. 1, the GUI 3000 discussed herein with reference to FIG. 3, the computing devices 4000 discussed herein with reference to FIG. 4, and/or the scientific instrument support system 5000 discussed herein with reference to FIG. 5), the method 2000 may be used in any suitable setting to perform any suitable support operations. Operations are illustrated once each and in a particular order in FIG. 2, but the operations may be reordered and/or repeated as desired and appropriate (e.g., different operations performed may be performed in parallel, as suitable).

At 2002, first operations may be performed. For example, the sample receiving logic 1002 of the support module 1000 may perform the operations of 2002. The first operations may include receiving a sample, from a queue, that includes an analyte.

At 2004, second operations may be performed. For example, method and parameter determining logic 1004 of the support module 1000 may perform the operations of 2004. The second operations may include determining instrument methods and data analysis parameters based on a nature of the analyte.

At 2006, third operations may be performed. For example, the processing logic 1006 of the support module 1000 may perform the operations of 2006. The third operations may include processing the sample by applying the instrument methods and data analysis parameters for an elution time where the mass is predicted as a function of elution time.

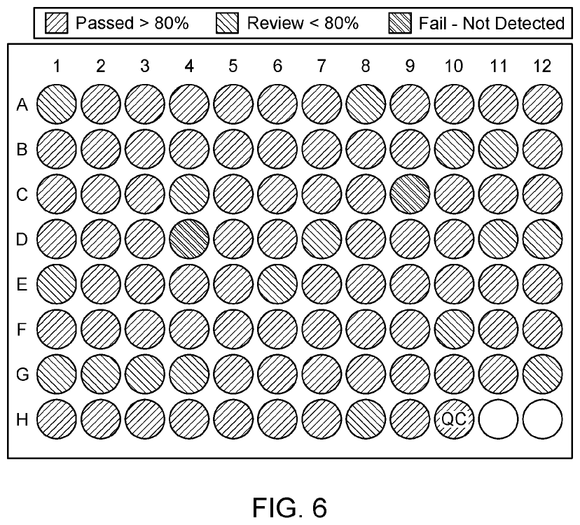

As noted above, in some embodiments, the data analysis performed on the instrument data may generate assessments of sample quality and/or sample purity. In some embodiments, a stop light color system (e.g., red, yellow, green, or other color or visual indicator) may be employed, in addition to or instead of numerical quality metrics, to mark sample quality in an easy to consume visual. FIG. 6 is an example of a visual indicator marking sample quality determinations generated using the instrument control and data analysis techniques disclosed herein for an array of samples disposed in wells of a tray. Although FIG. 6 is shown in varying levels of gray for ease of image reproduction, a stop light color system (red, yellow, green) may be used analogously.

The scientific instrument support methods disclosed herein may include interactions with a human user (e.g., via the user local computing device 5020 discussed herein with reference to FIG. 5). These interactions may include providing information to the user (e.g., information regarding the operation of a scientific instrument such as the scientific instrument 5010 of FIG. 5, information regarding a sample being analyzed or other test or measurement performed by a scientific instrument, information retrieved from a local or remote database, or other information) or providing an option for a user to input commands (e.g., to control the operation of a scientific instrument such as the scientific instrument 5010 of FIG. 5, or to control the analysis of data generated by a scientific instrument), queries (e.g., to a local or remote database), or other information. In some embodiments, these interactions may be performed through a graphical user interface (GUI) that includes a visual display on a display device (e.g., the display device 4010 discussed herein with reference to FIG. 4) that provides outputs to the user and/or prompts the user to provide inputs (e.g., via one or more input devices, such as a keyboard, mouse, trackpad, or touchscreen, included in the other I/O devices 4012 discussed herein with reference to FIG. 4). The scientific instrument support systems disclosed herein may include any suitable GUIs for interaction with a user.

FIG. 3 depicts an example GUI 3000 that may be used in the performance of some or all of the support methods disclosed herein, in accordance with various embodiments. As noted above, the GUI 3000 may be provided on a display device (e.g., the display device 4010 discussed herein with reference to FIG. 4) of a computing device (e.g., the computing device 4000 discussed herein with reference to FIG. 4) of a scientific instrument support system (e.g., the scientific instrument support system 5000 discussed herein with reference to FIG. 5), and a user may interact with the GUI 3000 using any suitable input device (e.g., any of the input devices included in the other I/O devices 4012 discussed herein with reference to FIG. 4) and input technique (e.g., movement of a cursor, motion capture, facial recognition, gesture detection, voice recognition, actuation of buttons, etc.).

The GUI 3000 may include a data display region 3002, a data analysis region 3004, a scientific instrument control region 3006, and a settings region 3008. The particular number and arrangement of regions depicted in FIG. 3 is simply illustrative, and any number and arrangement of regions, including any desired features, may be included in a GUI 3000.

The data display region 3002 may display data generated by a scientific instrument (e.g., the scientific instrument 5010 discussed herein with reference to FIG. 5). The data analysis region 3004 may display the results of data analysis (e.g., the results of analyzing the data illustrated in the data display region 3002 and/or other data). For example, results of the processing logic 1006 of the support module 1000 may provide to a user. In some embodiments, the data display region 3002 and the data analysis region 3004 may be combined in the GUI 3000 (e.g., to include data output from a scientific instrument, and some analysis of the data, in a common graph or region).

The scientific instrument control region 3006 may include options that allow the user to control a scientific instrument (e.g., the scientific instrument 5010 discussed herein with reference to FIG. 5). For example, the data display region 3002 may provide to a user an option to select a sample from a queue for the receiving logic 1002 of the support module 1000.

The settings region 3008 may include options that allow the user to control the features and functions of the GUI 3000 (and/or other GUIs) and/or perform common computing operations with respect to the data display region 3002 and data analysis region 3004 (e.g., saving data on a storage device, such as the storage device 4004 discussed herein with reference to FIG. 4, sending data to another user, labeling data, and the like).

As noted above, the scientific instrument support module 1000 may be implemented by one or more computing devices. FIG. 5 is a block diagram of a computing device 4000 that may perform some or all of the scientific instrument support methods disclosed herein, in accordance with various embodiments. In some embodiments, the scientific instrument support module 1000 may be implemented by a single computing device 4000 or by multiple computing devices 4000. Further, as discussed below, a computing device 4000 (or multiple computing devices 4000) that implements the scientific instrument support module 1000 may be part of one or more of the scientific instrument 5010, the user local computing device 5020, the service local computing device 5030, or the remote computing device 5040 of FIG. 5.

The computing device 4000 of FIG. 4 is illustrated as having a number of components, but any one or more of these components may be omitted or duplicated, as suitable for the application and setting. In some embodiments, some or all of the components included in the computing device 4000 may be attached to one or more motherboards and enclosed in a housing (e.g., including plastic, metal, and/or other materials). In some embodiments, some these components may be fabricated onto a single system-on-a-chip (SoC) (e.g., an SoC may include one or more processing devices 4002 and one or more storage devices 4004). Additionally, in various embodiments, the computing device 4000 may not include one or more of the components illustrated in FIG. 4, but may include interface circuitry (not shown) for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 4000 may not include a display device 4010, but may include display device interface circuitry (e.g., a connector and driver circuitry) to which a display device 4010 may be coupled.

The computing device 4000 may include a processing device 4002 (e.g., one or more processing devices). As used herein, the term “processing device” may refer to any device or portion of a device that processes electronic data from registers and/or memory to transform that electronic data into other electronic data that may be stored in registers and/or memory. The processing device 4002 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The computing device 4000 may include a storage device 4004 (e.g., one or more storage devices). The storage device 4004 may include one or more memory devices such as random access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 4004 may include memory that shares a die with a processing device 4002. In such an embodiment, the memory may be used as cache memory and may include embedded dynamic random access memory (eDRAM) or spin transfer torque magnetic random access memory (STT-MRAM), for example. In some embodiments, the storage device 4004 may include non-transitory computer readable media having instructions thereon that, when executed by one or more processing devices (e.g., the processing device 4002), cause the computing device 4000 to perform any appropriate ones of or portions of the methods disclosed herein.

The computing device 4000 may include an interface device 4006 (e.g., one or more interface devices 4006). The interface device 4006 may include one or more communication chips, connectors, and/or other hardware and software to govern communications between the computing device 4000 and other computing devices. For example, the interface device 4006 may include circuitry for managing wireless communications for the transfer of data to and from the computing device 4000. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not. Circuitry included in the interface device 4006 for managing wireless communications may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some embodiments, circuitry included in the interface device 4006 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 4006 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 4006 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 4006 may include one or more antennas (e.g., one or more antenna arrays) to receipt and/or transmission of wireless communications.

In some embodiments, the interface device 4006 may include circuitry for managing wired communications, such as electrical, optical, or any other suitable communication protocols. For example, the interface device 4006 may include circuitry to support communications in accordance with Ethernet technologies. In some embodiments, the interface device 4006 may support both wireless and wired communication, and/or may support multiple wired communication protocols and/or multiple wireless communication protocols. For example, a first set of circuitry of the interface device 4006 may be dedicated to shorter-range wireless communications such as Wi-Fi or Bluetooth, and a second set of circuitry of the interface device 4006 may be dedicated to longer-range wireless communications such as global positioning system (GPS), EDGE, GPRS, CDMA, WiMAX, LTE, EV-DO, or others. In some embodiments, a first set of circuitry of the interface device 4006 may be dedicated to wireless communications, and a second set of circuitry of the interface device 4006 may be dedicated to wired communications.

The computing device 4000 may include battery/power circuitry 4008. The battery/power circuitry 4008 may include one or more energy storage devices (e.g., batteries or capacitors) and/or circuitry for coupling components of the computing device 4000 to an energy source separate from the computing device 4000 (e.g., AC line power).

The computing device 4000 may include a display device 4010 (e.g., multiple display devices). The display device 4010 may include any visual indicators, such as a heads-up display, a computer monitor, a projector, a touchscreen display, a liquid crystal display (LCD), a light-emitting diode display, or a flat panel display.

The computing device 4000 may include other input/output (I/O) devices 4012. The other I/O devices 4012 may include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device 4000, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device 4000 may have any suitable form factor for its application and setting, such as a handheld or mobile computing device (e.g., a cell phone, a smart phone, a mobile internet device, a tablet computer, a laptop computer, a netbook computer, an ultrabook computer, a personal digital assistant (PDA), an ultra mobile personal computer, etc.), a desktop computing device, or a server computing device or other networked computing component.

One or more computing devices implementing any of the scientific instrument support modules or methods disclosed herein may be part of a scientific instrument support system. FIG. 5 is a block diagram of an example scientific instrument support system 5000 in which some or all of the scientific instrument support methods disclosed herein may be performed, in accordance with various embodiments. The scientific instrument support modules and methods disclosed herein (e.g., the scientific instrument support module 1000 of FIG. 1 and the method 2000 of FIG. 2) may be implemented by one or more of the scientific instrument 5010, the user local computing device 5020, the service local computing device 5030, or the remote computing device 5040 of the scientific instrument support system 5000.

Any of the scientific instrument 5010, the user local computing device 5020, the service local computing device 5030, or the remote computing device 5040 may include any of the embodiments of the computing device 4000 discussed herein with reference to FIG. 4, and any of the scientific instrument 5010, the user local computing device 5020, the service local computing device 5030, or the remote computing device 5040 may take the form of any appropriate ones of the embodiments of the computing device 4000 discussed herein with reference to FIG. 4.

The scientific instrument 5010, the user local computing device 5020, the service local computing device 5030, or the remote computing device 5040 may each include a processing device 5002, a storage device 5004, and an interface device 5006. The processing device 5002 may take any suitable form, including the form of any of the processing devices 4002 discussed herein with reference to FIG. 4, and the processing devices 5002 included in different ones of the scientific instrument 5010, the user local computing device 5020, the service local computing device 5030, or the remote computing device 5040 may take the same form or different forms. The storage device 5004 may take any suitable form, including the form of any of the storage devices 4004 discussed herein with reference to FIG. 4, and the storage devices 5004 included in different ones of the scientific instrument 5010, the user local computing device 5020, the service local computing device 5030, or the remote computing device 5040 may take the same form or different forms. The interface device 5006 may take any suitable form, including the form of any of the interface devices 4006 discussed herein with reference to FIG. 4, and the interface devices 5006 included in different ones of the scientific instrument 5010, the user local computing device 5020, the service local computing device 5030, or the remote computing device 5040 may take the same form or different forms.

The scientific instrument 5010, the user local computing device 5020, the service local computing device 5030, and the remote computing device 5040 may be in communication with other elements of the scientific instrument support system 5000 via communication pathways 5008. The communication pathways 5008 may communicatively couple the interface devices 5006 of different ones of the elements of the scientific instrument support system 5000, as shown, and may be wired or wireless communication pathways (e.g., in accordance with any of the communication techniques discussed herein with reference to the interface devices 4006 of the computing device 4000 of FIG. 4). The particular scientific instrument support system 5000 depicted in FIG. 5 includes communication pathways between each pair of the scientific instrument 5010, the user local computing device 5020, the service local computing device 5030, and the remote computing device 5040, but this “fully connected” implementation is simply illustrative, and in various embodiments, various ones of the communication pathways 5008 may be absent. For example, in some embodiments, a service local computing device 5030 may not have a direct communication pathway 5008 between its interface device 5006 and the interface device 5006 of the scientific instrument 5010, but may instead communicate with the scientific instrument 5010 via the communication pathway 5008 between the service local computing device 5030 and the user local computing device 5020 and the communication pathway 5008 between the user local computing device 5020 and the scientific instrument 5010.

The user local computing device 5020 may be a computing device (e.g., in accordance with any of the embodiments of the computing device 4000 discussed herein) that is local to a user of the scientific instrument 5010. In some embodiments, the user local computing device 5020 may also be local to the scientific instrument 5010, but this need not be the case; for example, a user local computing device 5020 that is in a user's home or office may be remote from, but in communication with, the scientific instrument 5010 so that the user may use the user local computing device 5020 to control and/or access data from the scientific instrument 5010. In some embodiments, the user local computing device 5020 may be a laptop, smartphone, or tablet device. In some embodiments the user local computing device 5020 may be a portable computing device.

The service local computing device 5030 may be a computing device (e.g., in accordance with any of the embodiments of the computing device 4000 discussed herein) that is local to an entity that services the scientific instrument 5010. For example, the service local computing device 5030 may be local to a manufacturer of the scientific instrument 5010 or to a third-party service company. In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to receive data regarding the operation of the scientific instrument 5010, the user local computing device 5020, and/or the remote computing device 5040 (e.g., the results of self-tests of the scientific instrument 5010, calibration coefficients used by the scientific instrument 5010, the measurements of sensors associated with the scientific instrument 5010, etc.). In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to transmit data to the scientific instrument 5010, the user local computing device 5020, and/or the remote computing device 5040 (e.g., to update programmed instructions, such as firmware, in the scientific instrument 5010, to initiate the performance of test or calibration sequences in the scientific instrument 5010, to update programmed instructions, such as software, in the user local computing device 5020 or the remote computing device 5040, etc.). A user of the scientific instrument 5010 may utilize the scientific instrument 5010 or the user local computing device 5020 to communicate with the service local computing device 5030 to report a problem with the scientific instrument 5010 or the user local computing device 5020, to request a visit from a technician to improve the operation of the scientific instrument 5010, to order consumables or replacement parts associated with the scientific instrument 5010, or for other purposes.

The remote computing device 5040 may be a computing device (e.g., in accordance with any of the embodiments of the computing device 4000 discussed herein) that is remote from the scientific instrument 5010 and/or from the user local computing device 5020. In some embodiments, the remote computing device 5040 may be included in a datacenter or other large-scale server environment. In some embodiments, the remote computing device 5040 may include network-attached storage (e.g., as part of the storage device 5004). The remote computing device 5040 may store data generated by the scientific instrument 5010, perform analyses of the data generated by the scientific instrument 5010 (e.g., in accordance with programmed instructions), facilitate communication between the user local computing device 5020 and the scientific instrument 5010, and/or facilitate communication between the service local computing device 5030 and the scientific instrument 5010.

In some embodiments, one or more of the elements of the scientific instrument support system 5000 illustrated in FIG. 5 may not be present. Further, in some embodiments, multiple ones of various ones of the elements of the scientific instrument support system 5000 of FIG. 5 may be present. For example, a scientific instrument support system 5000 may include multiple user local computing devices 5020 (e.g., different user local computing devices 5020 associated with different users or in different locations). In another example, a scientific instrument support system 5000 may include multiple scientific instruments 5010, all in communication with service local computing device 5030 and/or a remote computing device 5040; in such an embodiment, the service local computing device 5030 may monitor these multiple scientific instruments 5010, and the service local computing device 5030 may cause updates or other information may be “broadcast” to multiple scientific instruments 5010 at the same time. Different ones of the scientific instruments 5010 in a scientific instrument support system 5000 may be located close to one another (e.g., in the same room) or farther from one another (e.g., on different floors of a building, in different buildings, in different cities, etc.). In some embodiments, a scientific instrument 5010 may be connected to an Internet-of-Things (IoT) stack that allows for command and control of the scientific instrument 5010 through a web-based application, a virtual or augmented reality application, a mobile application, and/or a desktop application. Any of these applications may be accessed by a user operating the user local computing device 5020 in communication with the scientific instrument 5010 by the intervening remote computing device 5040. In some embodiments, a scientific instrument 5010 may be sold by the manufacturer along with one or more associated user local computing devices 5020 as part of a local scientific instrument computing unit 5012.

The following paragraphs provide various examples of the embodiments disclosed herein.

Example A1 is a mass spectrometry support apparatus including first logic to receive a sample from a queue, the sample comprising an analyte, second logic to determine instrument methods and data analysis parameters based on a nature of the analyte, and third logic to process the sample by applying the instrument methods and data analysis parameters for a mass spectrometry elution time where the mass is predicted as a function of elution time.

Example A2 includes the subject matter of Example A1, and further specifies that the nature of the analyte includes size, mass, shape, structure, or chemical composition.

Example A3 includes the subject matter of any of Examples A1 and A2, and further specifies that the sample is processed by coupling size-based separations.

Example A4 includes the subject matter of any of Examples A1-3, and further specifies that the parameters include hardware settings, ion optics, or detector parameters.

Example A5 includes the subject matter of any of Examples A1-4, and further specifies that the parameters are pre-optimized for a mass range and applied as an applied in-sync with the different masses coming off a column.

Example A6 includes the subject matter of any of Examples A1-5, and further specifies that the apparatus is a device for measuring the mass.

Example B1 is a scientific instrument support method, including: receiving, by a computing device, an indication of a mass of an analyte of interest, wherein the analyte of interest may be present in a sample; generating, by the computing device based at least in part on the mass indication, one or more instrument control parameters and one or more data processing parameters; providing, by the computing device, the one or more instrument control parameters for use by a mass spectrometer during analysis of the sample, wherein the mass spectrometer generates sample data based on the analysis of the sample; and providing, by the computing device, the one or more data processing parameters for use in processing the sample data.

Example B2 includes the subject matter of Example B1, and further includes: processing, by the computing device, the sample data using the one or more data processing parameters.

Example B3 includes the subject matter of Example B2, and further includes: causing, by the computing device, display of a visual indicator of sample quality or sample purity based on the processed sample data.

Example B4 includes the subject matter of Example B3, and further specifies that the visual indicator includes a stop light color system indicative of sample quality or sample purity.

Example B5 includes the subject matter of any of Examples B1-4, and further specifies that the data processing parameters include an indication of at least one of a noise filtering algorithm, a feature detection algorithm, a mass deconvolution algorithm, or a mass clustering algorithm.

Example B6 includes the subject matter of any of Examples B1-5, and further specifies that the data processing parameters include parameter values for a particular data processing algorithm.

Example B7 includes the subject matter of any of Examples B1-6, and further specifies that the instrument control parameters include at least one of hardware settings, ion optics, or detector parameters.

Example B8 includes the subject matter of any of Examples B1-7, and further specifies that receiving the indication of the mass of an analyte of interest includes receiving a user specification of a chemical structure or composition of the analyte.

Example B9 is a method for supporting cryo-electron microscopy (cryo-EM), including: receiving, by a computing device, an indication of a mass of an analyte of interest, wherein the analyte of interest may be present in a set of multiple samples; generating, by the computing device based at least in part on the mass indication, one or more instrument control parameters and one or more data processing parameters; causing analysis, by the computing device, of the samples in accordance with the instrument control parameters and the data analysis parameters; and identifying, by the computing device based at least in part on results of the sample analysis, one or more of the samples for further analysis by cryo-EM.

Example B10 includes the subject matter of Example B9, and further specifies that identifying the one or more samples for further analysis by cryo-EM includes causing the display of a visual indicator of one or more properties of the set of multiple samples.

Example B11 includes the subject matter of any of Examples B9-10, and further specifies that analysis of the samples includes coupling size-based separations.

Example B12 includes the subject matter of any of Examples B9-10, and further includes: causing, by the computing device, a vitrification process to be performed on the identified samples.

Example B13 is a method of performing a mass spectrometry process, including: receiving, by a computing device, an indication of a nature of an analyte of interest, wherein the analyte of interest may be present in a sample, and the nature of the analyte includes size, mass, shape, structure, or chemical composition; generating, by the computing device based at least in part on the nature indication, one or more instrument control parameters and one or more data processing parameters; and causing analysis, by the computing device, of the samples in accordance with the instrument control parameters and the data analysis parameters.

Example B14 includes the subject matter of Example B13, and further specifies that analysis of the samples includes size-based separations.

Example B15 includes the subject matter of any of Examples B13-14, and further specifies that the parameters are pre-optimized for a mass range and applied in-sync with the different masses coming off a column.

Example B16 includes the subject matter of any of Examples B13-14, and further includes: causing, by the computing device, display of a visual indicator of a property of the sample based on the analysis.

Example B17 includes the subject matter of Example B16, and further specifies that the visual indicator includes a stop light color system indicative of the property.

Example B18 includes the subject matter of any of Examples B13-17, and further specifies that the computing device is configured to generate one or more instrument control parameters and one or more data processing parameters for analytes of interest having a size between 0 kilodaltons and 1000 kilodaltons.

Example B19 includes the subject matter of any of Examples B13-18, and further specifies that analysis of the samples includes size exclusion chromatography or online buffer exchange.

Example B20 includes the subject matter of any of Examples B13-19, and further specifies that the analyte of interest is a protein.

1. A scientific instrument support method, comprising:
receiving, by a computing device, an indication of a mass of an analyte of interest, wherein the analyte of interest may be present in a sample;
generating, by the computing device based at least in part on the mass indication, one or more instrument control parameters and one or more data processing parameters;
providing, by the computing device, the one or more instrument control parameters for use by a mass spectrometer during analysis of the sample, wherein the mass spectrometer generates sample data based on the analysis of the sample; and
providing, by the computing device, the one or more data processing parameters for use in processing the sample data.

2. The scientific instrument support method of claim 1, further comprising:
processing, by the computing device, the sample data using the one or more data processing parameters.

3. The scientific instrument support method of claim 2, further comprising:
causing, by the computing device, display of a visual indicator of sample quality or sample purity based on the processed sample data.

4. The scientific instrument support method of claim 3, wherein the visual indicator includes a stop light color system indicative of sample quality or sample purity.

5. The scientific instrument support method of claim 1, wherein the data processing parameters include an indication of at least one of a noise filtering algorithm, a feature detection algorithm, a mass deconvolution algorithm, or a mass clustering algorithm.

6. The scientific instrument support method of claim 1, wherein the data processing parameters include parameter values for a particular data processing algorithm.

7. The scientific instrument support method of claim 1, wherein the instrument control parameters include at least one of hardware settings, ion optics, or detector parameters.

8. The scientific instrument support method of claim 1, wherein receiving the indication of the mass of an analyte of interest includes receiving a user specification of a chemical structure or composition of the analyte.

9. A method for supporting cryo-electron microscopy (cryo-EM), comprising:
receiving, by a computing device, an indication of a mass of an analyte of interest, wherein the analyte of interest may be present in a set of multiple samples;
generating, by the computing device based at least in part on the mass indication, one or more instrument control parameters and one or more data processing parameters;
causing analysis, by the computing device, of the samples in accordance with the instrument control parameters and the data analysis parameters; and
identifying, by the computing device based at least in part on results of the sample analysis, one or more of the samples for further analysis by cryo-EM.

10. The method of claim 9, wherein identifying the one or more samples for further analysis by cryo-EM includes causing the display of a visual indicator of one or more properties of the set of multiple samples.

11. The method of claim 9, wherein analysis of the samples includes coupling size-based separations.

12. The method of claim 9, further comprising:
causing, by the computing device, a vitrification process to be performed on the identified samples.

13. A method of performing a mass spectrometry process, comprising:
receiving, by a computing device, an indication of a nature of an analyte of interest, wherein the analyte of interest may be present in a sample, and the nature of the analyte includes size, mass, shape, structure, or chemical composition;
generating, by the computing device based at least in part on the nature indication, one or more instrument control parameters and one or more data processing parameters; and
causing analysis, by the computing device, of the samples in accordance with the instrument control parameters and the data analysis parameters.

14. The method of claim 13, wherein analysis of the samples includes size-based separations.

15. The method of claim 13, wherein the parameters are pre-optimized for a mass range and applied in-sync with the different masses coming off a column.

16. The method of claim 13, further comprising:
causing, by the computing device, display of a visual indicator of a property of the sample based on the analysis.

17. The method of claim 16, wherein the visual indicator includes a stop light color system indicative of the property.

18. The method of claim 13, wherein the computing device is configured to generate one or more instrument control parameters and one or more data processing parameters for analytes of interest having a size between 0 kilodaltons and 1000 kilodaltons.

19. The method of claim 13, wherein analysis of the samples includes size exclusion chromatography or online buffer exchange.

20. The method of claim 13, wherein the analyte of interest is a protein.
